(19) United States
(12) Patent Application Publication
(10) Pub. No.: US 2023/0037200 A1
(43) Pub. Date: Feb. 2, 2023

(54) 3D-Printed Implants And Methods For 3D Printing Of Implants

(71) Applicant: DePuy Synthes Products, Inc., Raynham, MA (US)

(72) Inventors: Fionnán Aodhán McNamara, Rathvilly County Carlow (IE); Sean Saidha, Franklin, MA (US); David Gerber, Providence, RI (US); Roderick McMillan, Downingtown, PA (US); Patrick A. Troy, Cork (IE); Edward Patrick Kavanagh, Cork (IE)

(21) Appl. No.: 17/861,165

(22) Filed: Jul. 8, 2022

Related U.S. Application Data
(60) Provisional application No. 63/226,212, filed on Jul. 28, 2021.

(51) Int. Cl. B22F 10/28; B23K 26/342; B28B 1/00; B33Y 10/00; B33Y 80/00; A61F 2/44

(52) U.S. Cl. CPC B22F 10/28; B23K 26/342; B28B 1/001; B33Y 10/00; B33Y 80/00; A61F 2/4455; A61F 2002/3085

(57) ABSTRACT

A method of making an implantable device includes directing a projection of laser energy having a plurality of adjacent energy pixels on a build surface atop a bed of powder, thereby forming a layer of the implantable device. The directing step is repeated a plurality of times, in a layer-by-layer manner, such that a totality of the formed layers define at least a portion of the implantable device.

### CROSS-REFERENCE TO RELATED APPLICATIONS

The present application claims the benefit of U.S. Provisional Application No. 63/226,212, filed Jul. 28, 2021, the entire contents of which are incorporated herein by this reference.

### TECHNICAL FIELD

The present invention relates to additive manufacturing processes for making implants, and more particularly to additive manufacturing processes that utilize projections of laser energy to create sequential layers of an implant.

### BACKGROUND

Additive manufacturing by selective laser sintering or melting denotes a process whereby sequential fusion of powder layers is used to create a three-dimensional (3D) object. To begin, a thin powder layer is dispensed on a working table (frequently referred to as the “build platform”), so that at least one layer of powder forms a powder bed. Selected areas of the powder layer are then fused by exposure to a directed energy source, typically a laser beam. The exposure pattern of the laser beam thus forms a cross-section of the three-dimensional object. The part is built through consecutive fusion of so-formed cross-sections that are stacked in layer-by-layer fashion along a vertical direction, and between the fusion of each layer the build platform is incremented downward and a new layer of powder is deposited onto the build surface. The general process of laser powder-fusion additive manufacturing has become known by several terms including selective laser melting (SLM), selective laser sintering (SLS), and direct metal laser sintering (DMLS), which terms are encompassed by the term laser powder bed fusion (L-PBF). These processes have been applied to various metals, ceramics, polymers, alloys, and composites.

### SUMMARY

According to an embodiment of the present disclosure, a method of making an implantable device includes directing a projection of laser energy on a build surface atop a bed of powder, thereby forming a layer of the implantable device. The projection of laser energy comprises adjacent energy pixels that share common boundaries on the build surface. Each pixel has a respective power density that is substantially uniform on the build surface. The directing step is repeated a plurality of times, in a layer-by-layer manner, such that a totality of the formed layers define at least a portion of the implantable device.

According to another embodiment of the present disclosure, an implant has a body that defines dimensions along first, second, and third directions that are substantially perpendicular to each other. The body defines at least one edge having a stepped profile that includes segments that are observable in a reference plane at 50× magnification. The at least one edge is curved and/or oriented oblique with respect to at least one of the first, second, and third directions.

### BRIEF DESCRIPTION OF THE DRAWINGS

The foregoing summary, as well as the following detailed description of illustrative embodiments of the present application, will be better understood when read in conjunction with the appended drawings. For the purposes of illustrating the features of the present application, there is shown in the drawings illustrative embodiments. It should be understood, however, that the application is not limited to the precise arrangements and instrumentalities shown. In the drawings:

FIG. 1 is a schematic representation of a prior art apparatus for additive manufacturing of an implant;

FIG. 2 is a schematic representation of a prior art laser line source delivering a linear projection of laser energy onto a build surface of a powder bed of the apparatus illustrated in FIG. 1;

FIG. 3A-3B are schematic representations of example prior art projections of laser energy composed of laser energy pixels; FIG. 3A illustrates a linear pixel array of rectangular laser energy pixels; and FIG. 3B illustrates an areal pixel array of rectangular laser energy pixels arranged in multiple rows;

FIG. 4A is a schematic representation of the prior art linear projection of laser energy illustrated in FIG. 2 delivered to a layer of powder material in which some areas of the layer are selectively fused by the projection of laser energy while others remain unfused;

FIG. 4B is a schematic representation of a prior art areal projection of laser energy delivered to a layer of powder material in which some areas of the layer are selectively fused by the areal projection while others remain unfused;

FIGS. 5A-5C are schematic representations of a layer of powder material exposed to multiple prior art laser sources that project various shapes of laser energy onto the layer;

FIG. 5D is a schematic representation of a layer of powder material exposed to a laser source that delivers a projection of laser energy onto the layer, which also includes a deposition-type print head for depositing additional material onto the layer, according to an embodiment of the present disclosure;

FIG. 6 is a schematic representation of a prior art projection of laser energy delivered to a layer of powder material constructed of two different powder material;

FIG. 7A is a perspective view of a layer of a multi-component implant constructed by delivery of the linear projection of laser energy illustrated in FIG. 2 to a layer of powder material, according to an embodiment of the present disclosure;

FIG. 7B is an enlarged plan view of a portion of the multi-component implant indicated by dashed region 7B in FIG. 7A;

FIG. 7C is a further enlarged plan view of a sub-portion of the multi-component implant indicated by dashed region 7C in FIG. 7B;

FIG. 8A is a photographic image showing, at 50× magnification, a select portion of a solid cube sample, manufactured via laser powder bed fusion (L-PBF) according to an embodiment of the present disclosure; and

FIG. 8A is a cross-sectional image of another portion of the solid cube sample of FIG. 8A, shown at 700× magnification.

### DETAILED DESCRIPTION OF ILLUSTRATIVE EMBODIMENTS

The present disclosure can be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the scope of the present disclosure. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “approximately”, “about”, and “substantially”, as used herein with respect to dimensions, angles, ratios, and other geometries, takes into account manufacturing tolerances. Further, the terms “approximately”, “about”, and “substantially” can include 10% greater than or less than the stated dimension, ratio, or angle. Further, the terms “approximately”, “about”, and “substantially” can equally apply to the specific value stated.

It should be understood that, although the terms first, second, etc. may be used herein to describe various elements, these elements should not be limited by these terms. These terms are instead used to distinguish one element from another. For example, a first element could be termed a second element, and, similarly, a second element could be termed a first element without departing from the scope of the embodiments disclosed herein.

The embodiments disclosed herein pertain to techniques for additive manufacturing of implantable devices (also referred to herein as “implants”), particularly techniques using at least one projection of laser energy on a build surface atop a powder bed to melt, sinter, or otherwise transform select regions of the powder into one or more solid, monolithic, implantable constructs. These techniques, encompassed herein by the term “laser powder bed fusion” (L-PBF), can provide for the rapid manufacture of implants, including those with complex geometries, with less material waste than reductive manufacturing processes. These techniques can also provide fine control of material properties of a manufactured implant.

Referring now to FIGS. 1-6, various exemplary systems, devices, and instrumentalities will be described for performing the additive manufacturing techniques of the present disclosure. Many of these features are more fully described in the following references: U.S. Patent Publication No. 2020/0139487 A1, published May 7, 2020, in the name of El-Dasher et al. (“the '487 Reference”) (listed on the face of the publication as being owned by Lawrence Livermore National Security LLC, of Livermore, Calif. in the United States); U.S. Patent Publication No. 2017/0304894 A1, published Oct. 26, 2017, in the name of Buller (“the '894 Reference”); U.S. Pat. No. 10,688,722, issued Jun. 23, 2020, in the name of Buller et al. (“the '722 Reference”); U.S. Pat. No. 10,434,573, issued Oct. 8, 2019, in the name of Buller et al. (“the '573 Reference”); U.S. Pat. No. 10,611,092, issued Apr. 7, 2020, in the name of Buller et al. (“the '092 Reference”); U.S. Pat. No. 10,888,925, issued Jan. 12, 2021, in the name of Symeonidis et al. (“the '925 Reference”) (the '894, '722, '573, '092, and '925 References are listed on the face of their publications as being owned by Velo3D, Inc., of Campbell, Calif. in the United States); U.S. Pat. No. 10,759,084, issued Sep. 1, 2020, in the name of Sullivan et al. (“the '084 Reference”) (listed on the face of the patent as being owned by Oceanit Laboratories Inc., of Honolulu, Hi. in the United States); U.S. Pat. No. 10,875,094, issued Dec. 29, 2021, in the name of Feldmann et al. (“the '094 Reference”); U.S. Pat. No. 10,919,090, issued Feb. 16, 2021, in the name of Feldmann et al. (“the '090 Reference”), (both of the '094 and the '090 References are listed on the face of their patents as being owned by VulcanForms Inc. of Burlington, Mass. in the United States); U.S. Pat. No. 10,399,183, issued Sep. 3, 2019, in the name of Dallarosa et al. (“the '183 Reference”) (listed on the face of the patent as being owned by IPG Photonics Corp., of Oxford, Mass. in the United States); U.S. Patent Publication No. 2017/0144224 A1, published May 25, 2017, in the name of DeMuth et al. (the '224 Reference) (listed on the face of the publication as being owned by Seurat Technologies, Inc., of Mountain View, Calif. in the United States); and International Patent Publication No. WO 2019/141381 A1, published Jul. 25, 2019, in the name of Stengel et al. (the '381 Reference) (listed on the face of the publication as being owned by SLM Solutions Group AG of Lubeck, Germany). The entire disclosures of each of these References are hereby incorporated by reference as if set forth in their entireties herein. It should be appreciated, however, that the aforementioned systems, devices, and instrumentalities are provided as non-limiting examples of hardware, processes, methods, and techniques that can be employed for the additive manufacturing techniques for making implants of the present disclosure. Other systems, devices, and instrumentalities that employ laser energy to fuse constituent powder material into a solid implant structure are also within the scope of the present disclosure.

Referring to FIG. 1, an example apparatus 100 for additive manufacturing according to an embodiment of the present disclosure includes a working table 5 with a powder bed 4 located inside a chamber 2. The chamber 2 includes a window 3 that allows a top surface 7 (also referred to as a “build surface” 7) of the powder bed 4 to be exposed to a laser source 1. The laser source 1, or other components positioned in the optical path of the laser source, includes means to change position of the laser beam projection relative to the powder bed such as gantry systems and/or mirror-based systems which may include one or more mirror galvanometers, which may be placed within or outside of the chamber. Means of modulating and/or shaping of the laser energy that intersects with the powder bed 4, include beam modulation devices and light valves (e.g. Grating Light Valves and Planar Light Valves). Controlled energy delivery from the laser source allows for selective fusion within a top layer of the powder (also referred to herein as a “build layer”) upon localized heating and subsequent cooling. The working table is then lowered along a first or vertical direction Z, and a new powder layer is distributed on the top of the powder bed. The build surface 7, which is now defined by the new powder layer, is exposed to the laser source 1 for further selective fusion within the powder layer upon localized heating and subsequent cooling. These steps (e.g., lowering the working table, distributing a new powder layer atop the powder bed, and exposing the new powder layer to the laser source 1) are repeated, layer-by-layer, as needed. In this manner, a three-dimensional (3D) part is fabricated (i.e., additively manufactured) as a plurality of consecutively fused cross-sections in layer-by-layer fashion. It can be appreciated that the cross-sections may be, but need not be, planar. In this example embodiment, the powder layer is formed with a recoater system, including a spreader mechanism 8 that spreads powder from a vertically actuated powder cartridge 6 in the working table region. Alternative instrumentalities and methods for powder layer formation may include deposition of powder by a nozzle mechanism, inkjet deposition, electro-hydrodynamic deposition, or ultrasonic deposition.

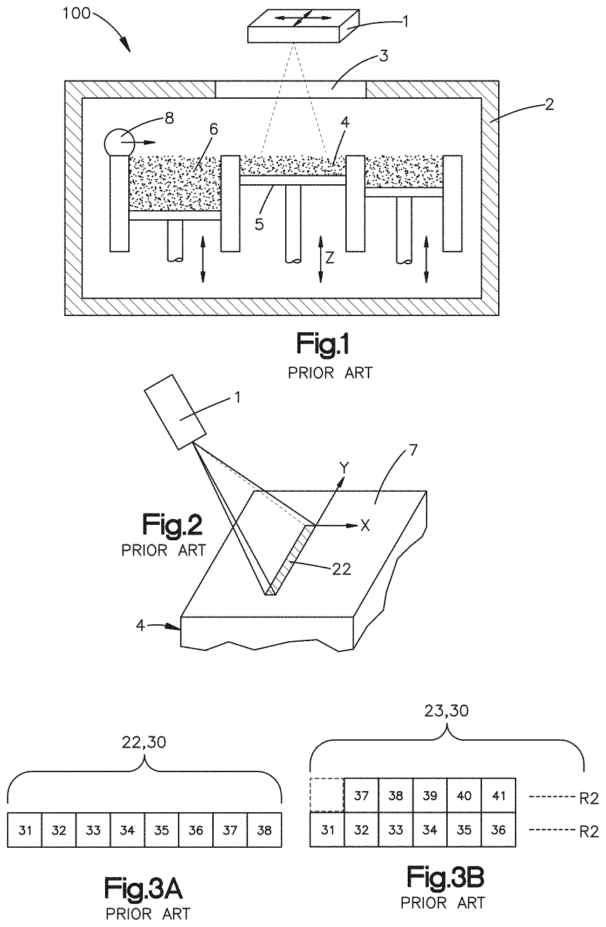

Referring now to FIG. 2, the laser source 1 is configured to deliver a projection of laser energy onto the build surface 7 (i.e., the top surface of the powder layer) for selectively fusing powder particles within one or more discrete regions of the powder layer. In the present embodiment, the laser source 1 is configured to deliver a linear projection 22 of laser energy. However, it should be appreciated that the depicted linear projection 22 represents a non-limiting example of the types of projections of laser energy producible by the laser sources disclosed herein. The laser source 1 can be configured to deliver various other projections of laser energy, such as areal projections 23, dot-shaped projections 12, or various combinations of the foregoing, as described in more detail below.

With continued reference to the embodiment shown in FIG. 2, the laser source 1 includes a control mechanism configured to control parameters of the linear projection 22, including an energy intensity profile thereof and spatial movement (e.g, scanning direction and scanning speed) of the linear projection 22 along the build surface 7. The intensity profile of the linear projection 22 can be adjusted as needed depending on the desired characteristics of the part being manufactured (e.g., material composition, infill density, moduli, by way of non-limiting examples). For example, the intensity profile of the linear projection 22 can be modulated along the direction of linear elongation, which in this example is denoted by second direction Y, which is substantially perpendicular to the first direction Z shown in FIG. 1. Additionally or alternatively, the intensity profile of the linear projection 22 can be modulated along both the elongation direction Y and along a transverse direction, which in this example is denoted by third direction X, which is substantially perpendicular to the first and second directions Y. The intensity profile of the linear projection 22 can be changed (e.g., along the elongation direction Y and/or along the transverse direction X), as needed, while fusing a single layer, between substantially uniform and non-uniform. For example, the parameters of the intensity can be chosen so that substantially an entirety of the linear projection 22 causes local fusion of the underlying powder on the build surface 7. Alternatively, the parameters of the intensity can be chosen so that one or more regions of the linear projection 22 cause local fusion of the underlying powder while one or more other regions of the linear projection 22 have an intensity below a fusion threshold, so that the underlying powder is not fused at these other region(s), thus achieving selective fusion of the powder within separated areas of the linear projection 22. It should be appreciated that the intensity profile(s) of the linear projection can be adapted as more fully described in the '090 Reference.

The control mechanism of the laser source 1 can employ one or more various control devices to adjust or otherwise modulate the intensity profile of the linear projection 22. By way of non-limiting examples, such control devices can include light valves, such as a grating light valve (GLV) to modulate the intensity along the line or a planar light valve (PLV) to modulate the intensity both along and across the line. Other means of spatial light modulation can be employed, such as intersecting the laser with a medium having locally tunable optical transmission, so only a portion of the laser energy, in a desired spatial pattern, is transmitted through the medium and incident upon the build surface.

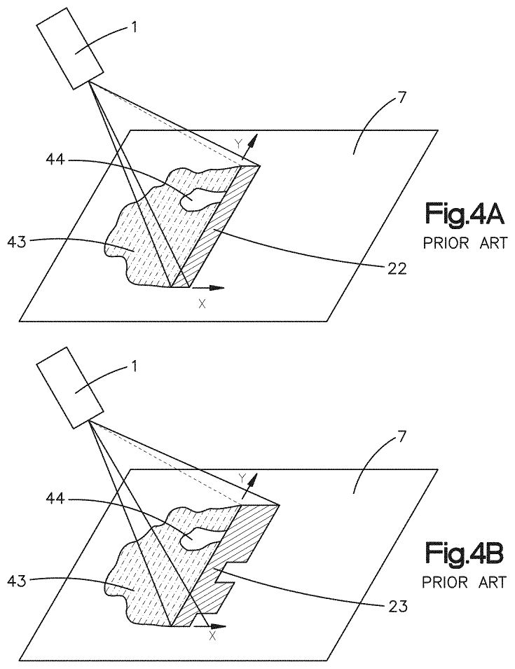

Examples of projections of laser energy for fusing powder bed particles will now be described with reference to FIGS. 3A-3B. In particular, projections of laser energy can be comprised of various arrays 30 of laser energy pixels. Aspects of these laser energy pixels can be controlled individually and/or collectively for controlling the intensity of the projection of laser energy.

Referring now to FIG. 3A, one such example embodiment for controlling the intensity of the linear projection 22 can include employing a linear array 30 of rectangular laser energy pixels 31-38, which pixels 31-38 combine to define the linear projection 22. In this manner, the linear projection 22 can comprise multiple individual laser energy pixels that are arranged adjacent to each other along the linear elongation direction Y. Although FIG. 3A shows eight (8) laser energy pixels 31-38, for illustrative purposes, it should be appreciated that the linear array 30 can include more than ten (10) pixels, more than 100 pixels, and more than 1000 pixels. Adjacent pixels 31-38 in the linear array 30 can share common boundaries with each other on the build surface 7, such that the power level (intensity) is substantially uniform along the linear projection 22 when each pixel is turned on.

The power density across any single pixel can be substantially uniform such that the pixel has a square shaped or “top hat” shaped energy profile when that pixel is turned on. The laser energy pixels 31-38 can have their respective power levels individually controlled and can each be turned on or turned off (i.e., iterated between an ON state and an OFF state) independently. In other embodiments, the power density across any single pixel can optionally vary according to various other energy profiles when that pixel is turned on. It should be appreciated that various means and instrumentalities for providing the laser energy pixels 31-38 and modulating their intensities can be provided, such as those more fully described in the '094 Reference, by way of a non-limiting example.

Referring now to FIG. 3B, in another example embodiment, a projection 23 of laser energy can employ an areal array 30 of laser energy pixels 31-41, which pixels can be rectangular, as described above. As shown, the areal array 30 can include a pixel grid having multiple rows R1, R2 of pixels. As above, the pixels in the areal array 30 can be individually controllable, such that various pixels therein can be iterated between an ON state and an OFF state (the OFF state is indicated in FIG. 3B by a dashed pixel border). It should be appreciated that various features of the arrays of laser energy pixels can be adapted as needed.

It should be appreciated that yet other means for directing, and/or modulating the intensity of, a linear projection of laser energy onto a build surface are within the scope of the present disclosure.

Referring now to FIGS. 4A-4B, it should also be appreciated that the intensity profile, scanning speed, and/or scanning direction applied to the projections described above, including the linear projection 22 (FIG. 4A) and the areal projection 23 (FIG. 4B), can each be simultaneously modulated as needed to create a desired spatial and temporal intensity patterns on the build surface 7. For example, the intensity profiles of the linear and areal projections 22, 23 can be modulated in such a fashion that not only the outer shape of a fused area 43 is controlled by the process but also so that any desired pattern of fused areas 43 and unfused areas 44 can be created within a powder layer. The scanning (i.e., translation) of the projections 22, 23 need not occur at uniform speed and can optionally follow various pathways across the build surface 7, such as pathways that alternate back and forth during the scanning of a layer, by way of a non-limiting example. The projections 22, 23 can be scanned at various speeds and along various paths, such as those more fully described in the '894 and '090 References, by way of non-limiting examples.

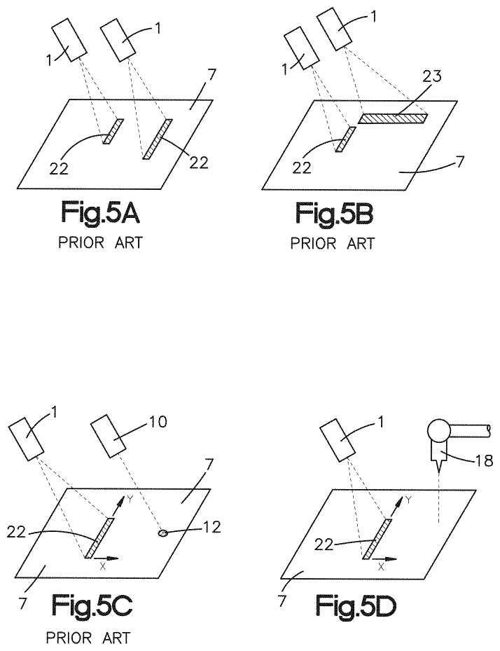

Referring now to FIGS. 5A-5D, the additive manufacturing apparatus can employ various combinations of laser sources 1, which can operate simultaneously and/or sequentially to fuse respective regions of powder in a build layer. For example, as shown in FIG. 5A, two (2) linear projection 22 laser sources 1 can be employed together such that the linear projections 22 are parallel with each other. Each such laser source 1 can be controlled independently on a build layer. As shown in FIG. 5B, a linear projection 22 laser source 1 and an areal projection 23 laser source 1 can be employed together such that their directions of elongation are angularly offset from each other, such as at a perpendicular orientation, although other angular offsets are within the scope of the present disclosure. As shown in FIG. 5C, a linear projection 22 laser source 1 can be employed with a laser source 10 that delivers a dot-shaped projection 12 on the build surface 7. As shown in FIG. 5D, in additional embodiments a linear projection 22 laser source 1 can be employed with a deposition 3D print head 18, which can operate sequentially and/or simultaneously on the build surface & (though preferably not simultaneously at the same region of the build surface 7). In this embodiment, the deposition 3D print head 18 can be employed for printing circuitry or other electronic components on a build layer, as described in more detail below. It should be appreciated that the foregoing combinations of multiple laser sources 1 can be adjusted as needed. For example, any of the linear projection 22 laser sources shown in FIGS. 5A-5D can be substituted for an areal projection 23 laser source, and vice versa.

Referring now to FIG. 6, the additive manufacturing apparatus can be adapted to manufacture parts constructed of two (2) or more different materials. In such embodiments, the powder bed 4 can contain powders having different material compositions, such as for construction of a build surface 61 comprising spatial arrangements of at least two materials, such as powder material P1 and powder material P2, which can have different melting temperatures. In the present illustrated example, the build surface 61 has areas 64, 65, 67 comprised of powder material P1 and areas 63, 66 comprised of powder material P2. Areas 63-67 are scanned with at least one laser source 1, which in this example is shown delivering a linear projection 22 onto the build surface 7. In other embodiments, the at least one laser source 1 can deliver an areal projection 23, a dot-shaped projection 12, or various combinations of projections of laser energy. The exposure to the laser energy source at a given power and scanning speed can cause one but not both of the powder materials P1, P2 to fuse. As shown, the linear projection 22 can scan the build surface 7 in the positive X direction. The depicted build surface 7 on the left side of the linear projection 22, including areas 66 and 67, has already been exposed to the laser yet only area 66 comprised of powder material P2 was fused while area 67, comprised of powder material P1, was not fused due to the exposure. The part of the build surface 7 to the right of the linear projection 22 including areas 63 and 64 has not been exposed to the laser yet and is thusly not fused at any point depicted. Once this area is exposed to the laser, again only area 63 comprised of powder material P2 will be fused while area 64 comprised of powder material P1 will remain unfused.

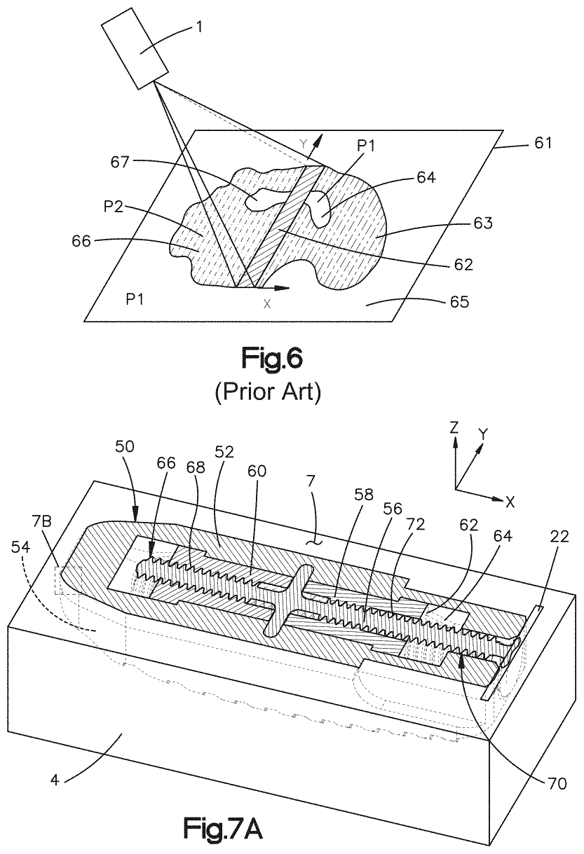

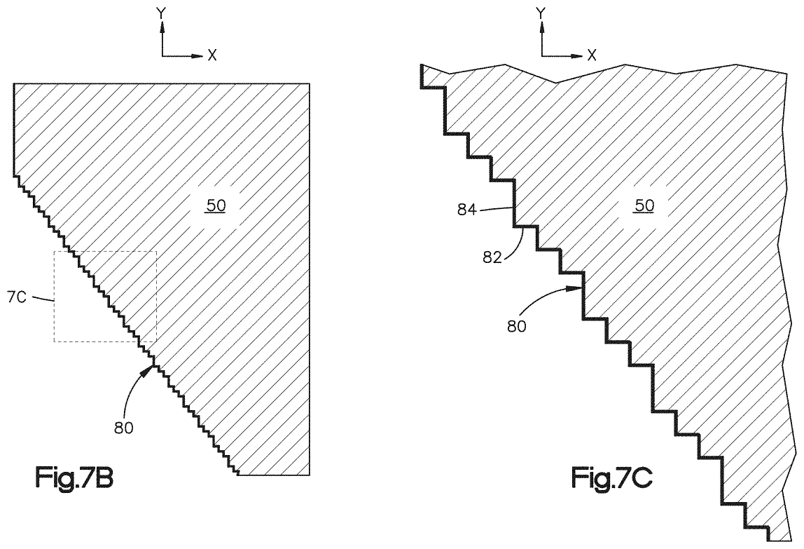

Referring now to FIG. 7A, an example of a multi-component implant 50 is shown during an intermediate step of manufacture according to the additive manufacturing techniques described herein. Although the depicted projection of laser energy in this example is a single linear projection 22, it should be appreciated that any of the projections of laser energy 12, 22, 23 described above, including combinations thereof, can be employed. The illustrated implant 50 is an intervertebral implant, particularly an intervertebral spinal fusion implant (also referred to as a “spinal fusion cage”), which is insertable into an intervertebral space between adjacent vertebral bodies while the implant is in a collapsed configuration. After insertion, the implant 50 is configured to expand along a cranial-caudal direction from the collapsed configuration to an expanded configuration, in which superior and inferior endplates 52, 54 of the implant engage the respective superior and inferior vertebral bodies. Thus, the illustrated implant 50 includes an expansion mechanism for increasing a distance between the endplates 52, 54 along the cranial-caudal direction. As shown, the expansion mechanism can include an actuator 56 that is drivable to actuate the expansion. The expansion mechanism of the illustrated embodiment also includes a pair of expansion wedges 58, 60 that have slide surfaces 62 that are configured to slide along complimentary guide surfaces 64 defined by the endplates 52, 54. The actuator 56 can be a shaft having one or more threaded portions, such as a first threaded portion 66 that defines threading 68 and a second threaded portion 70 that defines threading 72. The multi-component implant 50 can be manufactured in layer-by-layer fashion, with the respective components in their relative positions. To facilitate such construction, the apparatus 100 can include a multi-axis powder deposition head, which deposit the respective powder compositions in their relative positions for each build layer. In such embodiments, the linear projection 22 can be directed (i.e., scanned) on the build surface and/or modulated as needed to prevent separate components from fusing together in the build layer. It should be appreciated that interfacing and/or interconnected components can have spatial resolution and accuracy (and gap sizes) at scales less than about 10 μm.

Referring now to FIGS. 7B-7C, due to the use of the linear projection 22 and/or the areal projection 23 to form the layers of the implant 50, the edges 80 of the implant 50 viewed in a horizontal plane (i.e, the X-Y plane), particularly those edges 80 that are rounded or extend at an oblique angle with respect to the elongation direction Y or the transverse direction X, tend to have a jagged, stepped, pixel-like, or otherwise irregular non-smooth profile compared to that of a traditionally formed implant, at least when viewed under 50× magnification or greater, including magnifications in a range of 20× to 200×, for example. For illustrative purposes, FIGS. 7B-7C show such an irregular non-smooth profile as comprising linear sections 82, 84, although the irregular profile can have rounded portions (such as defined by non-melted powder particles affixed to adjacent melted material) or other micro-shaped features. Moreover, due to the layer-by-layer formation process, edges of the implant 50, particularly those edges that are rounded or extend at an oblique angle with respect to the vertical and transverse directions Z, X, will also show a similar irregular/stepped profile, at least when viewed under 50× magnification or greater, such as a range of 20× to 200× magnification. In some embodiments, such irregular/stepped profiles of rounded or oblique edges may not be observable at magnifications less than about 18× magnification.

Example methods of making an implantable device (also referred to herein as an “implant”) will now be described. It should be appreciated that the following example methods are provided as non-limiting example methods. Accordingly, other methods not specifically set forth below can be within the scope of the present disclosure.

A method of making an implant, according to one example, includes a step of directing a liner projection of laser energy and/or an areal projection of laser energy to a build surface atop a bed of powder, thereby fusing particles of the powder together in a manner forming a layer of an implant. The linear and/or areal projection can optionally be an array of energy pixels that are configured such that adjacent energy pixels therein share common boundaries on the build surface, and each pixel has a respective power density that is substantially uniform on the build surface. This example method includes steps of repeating the directing step a plurality of times, in a layer-by-layer manner, such that a totality of the formed layers define at least a portion of the implant. The repeating steps can be performed until, upon conclusion of a final one of the repeating steps, an entirety of the implant is formed.

Each repeating step includes lowering the powder bed 4 and spreading a new build layer of powder over the previously formed layer, such as with the spreader mechanism 8. At the conclusion of the repeating steps, unfused powder particles can be removed, such as via one or more vacuum nozzles.

As described above, the implant formed at the conclusion of the repeating steps has edges that define irregular/stepped profiles that are observable in a horizontal reference plane and/or a vertical reference plane when viewed under 50× magnification of greater. The magnification can be performed using light optical microscopy, by way of a non-limiting example.

The implant formed according to these example methods and steps can be an orthopedic implant, such as an intervertebral implant (e.g., an expandable spinal fusion cage), a vertebral body replacement (VBR) implant, or a bone plate fixation device (e.g., a cervical spinal implant), by way of non-limiting examples. It should be appreciated that numerous other types of orthopedic implants can be manufactured according to the additive manufacturing processes described herein, including, but not limited to, bone plates (e.g., rigid plates and articulable, interconnected link-type plates), bone anchors (e.g., bone screws), anchor heads, spinal rods, guide wires (e.g., K-wires), rigid suture anchors, suture hubs, reconstructed joints, platforms, intramedullary nails, synthetic bone graft, and the like. It should further be appreciated that the additive manufacturing processes described herein can be employed to manufacture instrumentation for assistance during an implantation procedure, such as insertion instruments, retractors, guide channels, trial spacers, tissue-cutting members, and bone graft delivery devices, by way of non-limiting examples. The implants herein can be configured for use in bone tissues (including cortical and/or cancellous tissue) and soft tissues (e.g., muscle, tendon, ligament, organs). Virtually any type of rigid implant or implant having rigid components can be manufactured according to the additive manufacturing processes described herein.

Based on, for example, implant type, one or more of the build layers can include separate components of the implant, which components can be interconnected within the build layer and/or across multiple layers. When separate components are interconnected within a build layer, the respective repeating step can be characterized as effectively interconnecting the components, or at least a layer-wise portion thereof, as described in more detail below.

It should be appreciated that the methods described herein allow in-layer interconnection of components that have macrostructure (e.g., dimensions from 1 mm to 200 mm or greater), microstructure (e.g., dimensions from 1 micrometer (μm) to 1000 micrometers (μm)), and nanostructure (e.g., dimensions under 100 μm). For example, in some embodiments, the interconnected components have spatial resolution and accuracy at scales less than about 10 μm. In further embodiments, nanostructures can be added to the powder bed, such as within one or more respective build layers. In such embodiments, the build layer can contain a powder base material and a second material comprising or consisting of nanostructures, such as carbon nanotube (CNT's) or nanoparticles, with at least one dimension in the range of 1-100 nm. It should be appreciated that such nanostructures need not differ in material composition from the base material of the powder, though the nanoparticles can have a suppressed melting/sintering temperature due to their size.

In some embodiments of the example structures and methods, an in-layer construct or a multi-layer construct can include a first interconnected component of the construct defines a guide surface that is sufficiently smooth to provide a sliding contact interface with a complimentary movement surface, such as a sliding surface, defined by at least a second interconnected component of the construct. The sliding contact interface can facilitate an actuation process of the implant during an implantation procedure, such as an expansion process of an expandable fusion cage, by way of a non-limiting example. In this manner, such complimentary guide/movement surfaces can be defined on various movable components of the implant, such as actuators, expansion members, securing and/or retention members (e.g., deployable spikes and/or barbs for engaging adjacent tissue, such as vertebrae), and/or locking members, by way of non-limiting examples. In some such embodiments, such as those involving an expandable fusion cage, the interconnected movable components can include expansion wedges having portions configured to slide along guide grooves or channels defined in one or both of the endplates. In further such embodiments, the interconnected movable components can include mating threads, such as external threads on an actuator configured to intermesh with internal threads within respective bores of the expansion wedges, by way of non-limiting examples. In yet additional such embodiments, the interconnected movable components can include locking members, such as locking pins that are deployable to affix a position of an actuator and/or an expansion member (e.g., expansion wedge), thereby affixing an expanded height of a fusion cage, by way of non-limiting examples. It should be appreciated that, in some embodiments of the structures and methods herein, the interconnected movable components can be functional or at least pseudo-functional at the conclusion of the build and removal of unfused powder, including without need of supplemental surface finishing processes.

Additionally or alternatively, portions of an in-layer construct or multi-layer constructs can define interior spaces such as voids and/or conduits through the implant or portions thereof. Such spaces, voids, and/or conduits can be configured for delivering, transmitting, receiving, and/or retaining bio-materials, such as bone graft, bone ingrowth inducing material, and the like, by way of non-limiting examples. Additionally or alternatively, such spaces, voids, and/or conduits can be configured for selective reception of movable components of the implant, such as those described above.

Additionally or alternatively, such spaces, voids, and/or conduits can be configured to receive electronic circuitry, such as printed circuit boards (PCBs), processors, microprocessors, computer memory, communication devices, sensors, and the like, by way of non-limiting examples. Such electronic circuitry can include “smart” electronic components, such as types configured to autonomously or semi-autonomously execute one or more algorithms, such as software or other computer programs. Such smart electronics can include one or more of an accelerometer, a strain gauge, a proximity sensor, a PH sensor, a thermal sensor, and a thermal conductor, by way of non-limiting examples. In such embodiments, the methods herein can include disposing preconstructed electronics within receptacles defined within a build-layer or across multiple build layers. Additionally or alternatively, the methods herein can include steps of making electrical components in a build-layer or in multiple build-layers. For example, such steps can include depositing, such as via printing, such as 3D printing, electronic circuitry on a build layer. The circuitry can be 3D printed by one or more deposition 3D print heads 18, such as that described above with reference to FIG. 5D. In such steps, a 3D print head can deposit a constituent layer of substrate material on a build-layer (e.g., an in-layer construct). The 3D print head or another 3D print head can subsequently deposit one or more additional layers on the substrate, such as a layer of semiconductor material and conductive traces within or along the semiconductor layer, by way of non-limiting examples.

The powder bed 4 can have various material compositions depending on the desired composition of the implant. The powder materials can include metals, ceramics, polymers, alloys, and composites. The materials can include medical-grade or otherwise biocompatible materials, and can optionally include non-biocompatible materials, such as in embodiments where such latter materials are encased or otherwise sealed within a biocompatible material in the built implant. By way of non-limiting examples, the metals can include stainless steels, construction steels, light metals and alloys (titanium, aluminum and aluminum-lithium alloys), additional alloys (titanium-aluminum-vanadium allows (e.g., TAV, such as Ti₆₄Al₄V, also referred to as Ti64), titanium-molybdenum alloys (e.g., TiMo), cobalt-chromium alloys (e.g., CoCr)), superalloys (e.g. nickel base alloys such as Inconel and Hastelloy), hard and refractory metals (e.g. tungsten and molybdenum), precious metals (e.g. gold), heat and electrically conductive metals (e.g., copper and silver). Ceramics may herein refer to, but are not limited to inorganic, non-metallic solids comprised of metallic, metalloid or non-metallic atoms. Examples are carbides, nitrides and borides (e.g. tungsten and titanium carbide, silicon nitride and carbide and boron nitride) as well as oxides such as aluminum oxide, zinc oxide and zirconia. Polymer may herein refer to, but are not limited to photopolymers, thermoplastics and thermosetting polymers.

In case of the material being applied to the build surface as powder, such powder particles can be of various sizes, size (and average size) distributions as well as different geometrical shapes. Powder size (and average size) distributions may range from 1-1000 nm, 1-100 μm, and/or 10 μm to 1 mm. The powder particle sizes can be selected based on sizes and material compositions conducive for favorable fusing and fused grain structure of the built implant.

Various build layers can contain multiple materials and powders, such as a combination of at least one metallic powder and at least one ceramic powder. In such embodiments, the laser energy (e.g., the linear and/or areal projections 22, 23, such as those comprising arrays of energy pixels) can be applied to a build layer to form at least one metallic component of the implantable device and concurrently form at least one ceramic component of the implantable device. For example, such use of metallic powders and ceramic powders can be employed with the laser energy in layer-by-layer fashion to produce an implant that includes at least one metallic component and at least one ceramic component or feature. For example, the built implant can include at least one metallic component that has a ceramic coating that coats at least a portion of the at least one metallic component. In such embodiments, the ceramic coating can be resorbable, such as a ceramic coating comprising hydroxyapatite (HA).

At least one of the in-layer constructs can have a fused microstructure that is substantially devoid of surface defects. In further embodiments, the build implant can include one or more microstructures each substantially devoid of surface defects or defects adjacent the surfaces. Moreover, such microstructures can have alpha martensitic grain structures at the conclusion of the build (e.g., before any post-build heat treatment), such as those of stainless steel, TAV, cobalt-chromium, and TiMo, by way of non-limiting examples. The methods herein can further include performing one or more surface finishing steps on one or more of the implant surfaces, such as to provide a surface finish roughness configured to promote osteogenesis.

The methods herein can further include performing one or more heat treating processes, such as vacuum thermal processes. Such thermal processes can facilitate and/or enhance dynamic properties of the built implant, such as its fatigue performance over the life of the implant. For example, post-build heat treatment processes can facilitate alpha martensitic grain structures in the implant or can enhance existing alpha martensitic grain structures of the implant. Vacuum thermal processes can be a preferred post-build thermal process because it can enhance dynamic properties without the need for hot isostatic pressing. It should be appreciated, however, that various other heat treating processes can be employed as needed, including hot isostatic pressing. The methods herein can yet also include optional post-build finishing processes, such as applying one or more various coatings or supplemental exterior layers to the implant.

Various aspects of the foregoing steps can be adapted as needed so that the built implant or at least a respective portion thereof has a targeted modulus of elasticity. In one such non-limiting example, select aspects of the foregoing steps can be adapted such that a first discrete region of a portion of the built implant has a first modulus of elasticity, and a second discrete region of the portion of the built implant has a second modulus of elasticity that differs from the first discrete region.

According to embodiments of the methods described above, after conclusion of the repeated steps (i.e., at the conclusion of the “build”), the implant (i.e., the “built implant”) or select portions thereof can have a printed infill density in a range of about 35 percent to 100 percent, which can vary as-desired at different regions of the implant. The solid portions of the built implant can also have a volume density in a range of about 99.5 percent to about 100 percent, and more particularly about 99.8 percent. The build implant can have a hardness in a range from about 32 HRC to about 40 HRC, as measured according to the Hardness Rockwell C (HRC) scale. It should be appreciated that the implant's printed density and/or hardness can be adapted, such as by adjusting the material composition, powder size, and infill volume, by way of non-limiting examples.

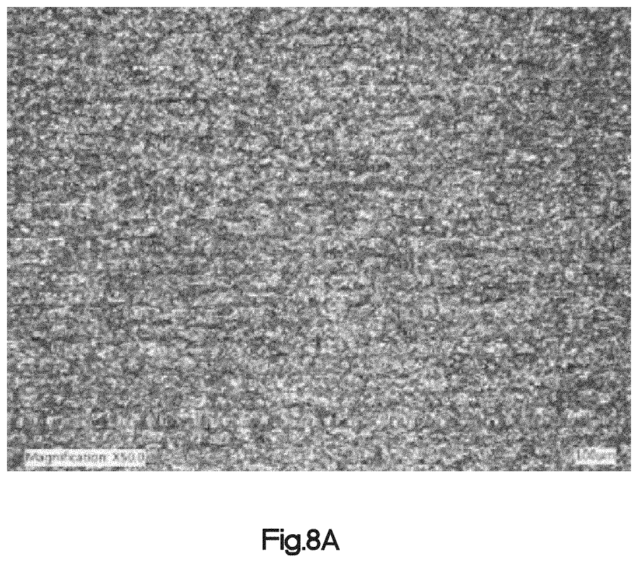

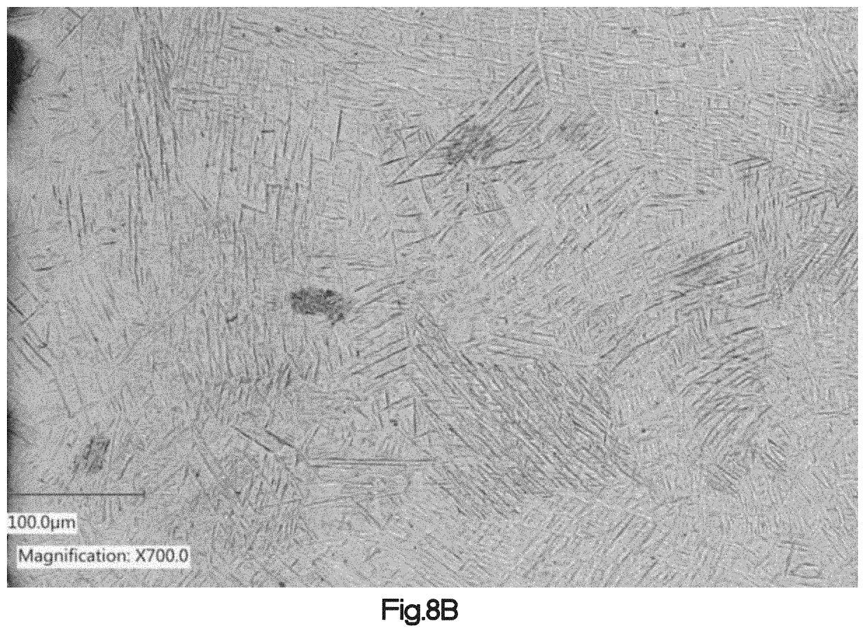

According to one such non-limiting example, the implant can be constructed by exposing a TAV powder (specifically, Ti₆₄Al₄V) to a linear and/or areal projection of laser energy in a layer-by-layer 3D-printing process utilizing a 100 percent infill volume. In one study of such 3D-printed TAV constructs, the inventors tested various 3D-printed samples of solid bodies having different shapes, each of these solid constructs printed from Ti₆₄Al₄V constituent powder. For comparison, the inventors also tested additional samples of similar shaped objects that were 3D-printed from the same constituent powder type (Ti₆₄Al₄V) but having a reduced infill volume (i.e., having open spaces therein). The 3D-printed and tested samples can be characterized as representing various components of an implant. The printed samples were imaged with a Keyence Light Optical System (Model VHX-5000) and a Zeiss EV060 XVP scanning electron microscope (SEM) for inspection of, among other things, sample porosity and microstructure. Various images of one of the samples are shown in FIGS. 8A-8B. The samples were also subjected to hardness tests.

The inventors made several surprising observations during these tests. For example, by analyzing cross-sections of the samples it was observed that the microstructures of the solid portions of the samples possessed a volume density (i.e., inverse of porosity) of about 99.8 percent. It was also observed that the last printed layers in all of the above-referenced samples exhibited non-melted spheres around the outer surfaces. No microcracks, burrs or any deformed material was observed in any of the samples. The samples each demonstrated a hardness in a range from 35-38 HRC and a 0% area porosity within the confines of the measurement systems. One advantage of the additive manufacturing processes described herein, as demonstrated by these test results, is that because the as-built porosity of the solid portions of the implant/components is near 0% porosity, the need for post-build processing to resolve defects can be avoided or at least reduced.

Referring now to FIG. 8A, a magnified light optical (LO) view of a side surface of the solid sample is shown at 50× magnification, revealing the layer-wise structure of the sample body. FIG. 8B is a cross-sectional image of the solid body sample at 700× magnification, which shows the sample's microstructure, demonstrating an alpha martensitic grain structure. All of the above-referenced samples possessed an alpha martensitic grain structure.

It should be appreciated that the methods and instrumentalities described above can advantageously be employed for rapid implant manufacture. It should also be appreciated that the rapidity of the manufacturing processes described herein can allow for rapid, on-demand production of an implant having a patient-specific geometry (i.e., an implant geometry tailored to correspond to the patient-specific anatomy in which the implant is configured to reside), which geometry can be based on part on scan data of patient anatomy. For example, in the case of a VBR implant, the implant geometry can be created in 3D virtual space with the assistance of patient scan data of the vertebral body to be replaced (such as a 3D model constructed with the assistance of a series of CT-scan slices of the vertebral body), which 3D virtual implant geometry can be tailored as needed to provide a treatment objective. In the case of an expandable spinal fusion cage, the implant geometry can be created in 3D virtual space with the assistance of scan data of the adjacent vertebral bodies. In particular, the opposed outer surfaces of the endplates can be tailored in 3D virtual space to have contours that correspond to those of the respective superior and inferior surface of the adjacent vertebral bodies. The cage geometry can be further tailored, for example, based on the desired post-operative intervertebral height. The rapid additive manufacturing methods described herein can be employed to create an implant possessing the tailored 3D virtual geometry based on patient-specific data.

It should be appreciated that the various parameters, properties, and characteristics of the materials, energy, and processes described above are provided as exemplary features for adapting the manufacturing processes and instrumentalities to 3D print or otherwise additively manufacture implants. These parameters can be adjusted as needed without departing from the scope of the present disclosure.

It should further be appreciated when a numerical preposition (e.g., “first”, “second”, “third”) is used herein with reference to an element, component, dimension, step, or a feature thereof (e.g., “first” portion, “second” portion, “first” step, “second” step), such numerical preposition is used to distinguish said element, component, dimension, step, and/or feature from another such element, component, dimension, step, and/or feature, and is not to be limited to the specific numerical preposition used in that instance. For example, a “first” portion, component, or step can also be referred to as a “second” portion, component, or step in a different context without departing from the scope of the present disclosure, so long as said portions, components, and/or steps remain properly distinguished in the context in which their numerical prepositions are used.

Although the disclosure has been described in detail, it should be understood that various changes, substitutions, and alterations can be made herein without departing from the spirit and scope of the invention as defined by the appended claims. Moreover, the scope of the present disclosure is not intended to be limited to the particular embodiments described in the specification. In particular, one or more of the features from the foregoing embodiments can be employed in other embodiments herein. As one of ordinary skill in the art will readily appreciate from that processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A method of making an implantable device, comprising:
directing a projection of laser energy on a build surface atop a bed of powder, thereby forming a layer of an implantable device, wherein the projection of laser energy comprises adjacent energy pixels that share common boundaries on the build surface, and each pixel has a respective power density that is substantially uniform on the build surface;
repeating the directing step a plurality of times, in a layer-by-layer manner, such that a totality of the formed layers define at least a portion of the implantable device.

2. The method of claim 1, wherein after the repeating step the at least a portion of the implantable device defines at least one edge having an irregular profile observable in a reference plane under 50× magnification.

3. The method of claim 2, wherein the 50× magnification is performable using light optical microscopy.

4. The method of claim 1, wherein the implantable device is an orthopedic device.

5. The method of claim 4, wherein the implantable device is a spinal fusion device.

6. The method of claim 5, wherein the directing and repeating steps comprise forming layers of respective endplates and an actuation mechanism of the expandable spinal fusion device, and the actuation mechanism is configured to expand a distance between the endplates after conclusion of the repeating steps.

7. The method of claim 1, wherein, after conclusion of the repeated steps, the implantable device has a printed density in a range of about 99.5 percent to about 100 percent.

8. The method of claim 7, wherein, after conclusion of the repeated steps, the implantable device has a hardness in a range from about 32 HRC to about 40 HRC.

9. The method of claim 1, wherein the projection of laser energy comprises at least one of a linear array of energy pixels and an areal array of energy pixels on the build surface, such that adjacent energy pixels in the at least one array share common boundaries on the build surface, and each pixel has a respective power density that is substantially uniform on the build surface.

10. The method of claim 1, wherein, during at least some of the repeated directing steps, the respective layer comprises portions of interconnectable components of the implantable device.

11. The method of claim 10, wherein the interconnectable components are interconnected during at least some of the repeating steps, and the interconnected components having at least one of macrostructure, microstructure, and nanostructure.

12. The method of claim 11, wherein the interconnected components have spatial resolution and accuracy at scales less than about 10 micrometers (μm).

13. The method of claim 11, wherein the interconnected components collectively define conduits through the implantable device.

14. The method of claim 10, wherein after the repeating steps, the totality of the formed layers define the implantable device having interconnected components.

15. The method of claim 14, wherein at least a first one of the interconnected components defines a guide surface that is sufficiently smooth to provide a sliding contact interface with a complimentary surface defined by at least a second one of the interconnected components during an actuation process of the implantable device in an implantation procedure.

16. The method of claim 14, wherein the interconnected components comprise deployable securing spikes for securing the implantable device to one or more vertebrae.

17. The method of claim 1, wherein the powder bed contains metallic powder, such that the implantable device comprises metal.

18. The method of claim 17, wherein the powder bed further contains ceramic powder, such that, during at least some of the repeating steps, the energy pixels form at least one metallic component of the implantable device and concurrently form at least one ceramic component of the implantable device, wherein, after conclusion of the repeating steps, the at least one ceramic component is a ceramic coating that coats at least a portion of the at least one metallic component.

19. The method of claim 18, wherein the ceramic coating is resorbable.

20. The method of claim 18, wherein the ceramic coating comprises hydroxyapatite (HA).

21. The method of claim 1, wherein, during at least some of the repeated directing steps, the respective layer comprises a microstructure substantially devoid of defects at or adjacent a surface of the implantable device.

22. The method of claim 21, wherein, after conclusion of the repeating steps, the implantable device comprises one or more microstructures each substantially devoid of defects at or adjacent a surface of the implantable device.

23. The method of claim 22, wherein the one or more microstructures is alpha martensitic after conclusion of the repeating steps.

24. The method of claim 23, wherein the one or more microstructures comprise one or more materials selected from the group of stainless steel, a titanium-aluminum-vanadium (TAV) alloy, a titanium-molybdenum alloy, and a cobalt-chromium alloy.

25. The method of claim 23, further comprising surface finishing the one or more microstructures to provide the one or more microstructures with a surface finish roughness configured to promote osteogenesis.

26. The method of claim 23, further comprising vacuum thermal processing the implantable device after conclusion of the repeating steps, wherein the vacuum thermal processing enhances fatigue performance of the implantable device.

27. The method of claim 1, wherein the directing and repeating steps are performed such that, after conclusion of the repeating steps, the at least a portion of the implantable implant has a targeted modulus of elasticity.

28. The method of claim 1, wherein the directing and repeating steps are performed such that, after conclusion of the repeating steps, a first discrete region of the at least a portion of the implantable implant has a first modulus of elasticity, and a second discrete region of the at least a portion of the implantable implant has a second modulus of elasticity that differs from the first discrete region.

29. The method of claim 1, wherein the implantable device is a vertebral body replacement device.

30. The method of claim 29, wherein the directing and repeating steps are performed after a vertebral corpectomy.

31. The method of claim 1, further comprising, during at least one of the repeated directing steps, printing electronic circuitry onto the respective layer.

32. The method of claim 31, wherein the step of printing electronic circuitry comprises depositing a substrate onto the layer and further depositing semiconductor material and conductive traces over the substrate.

33. The method of claim 31, wherein the electronic circuitry comprises smart electronics configured to execute one or more computer programs.

34. The method of claim 31, wherein the electronic circuitry comprises one or more of an accelerometer, a strain gauge, a proximity sensor, a PH sensor, a thermal sensor, and a thermal conductor.

35. The method of claim 1, wherein after the repeating steps, the totality of the formed layers define the implantable device, wherein the implantable device has voids for delivering and receiving bone graft.

36. The method of claim 1, wherein after the repeating steps, the totality of the formed layers define the implantable device, wherein the implantable device has functional threads.

37. An implant, comprising:
a body defining dimensions along a first direction, a second direction, and a third direction, wherein the first, second, and third directions are substantially perpendicular to each other,
wherein the body defines at least one edge having a stepped profile comprising segments that are observable in a reference plane at 50× magnification, wherein the at least one edge is one or both of curved and oriented oblique with respect to at least one of the first, second, and third directions.

38. The implant of claim 37, wherein the stepped profile is substantially not observable at magnifications less than 18× magnification.

39. The implant of claim 37, wherein the body comprises a plurality of layers spaced in series along the third direction, and at least two adjacent layers of the plurality of layers have a stepped geometry with respect to each other, wherein the stepped geometry is observable under 50× magnification.

40. The implant of claim 37, wherein a majority of the body is constructed of a material that consists essentially of fused particles.

41. The implant of claim 40, wherein the fused particles are melted.

42. The implant of claim 40, wherein the fused particles are melted together such that interstitial voids extend between a majority of the fused particles.
